(19) United States
(12) Patent Application Publication
(10) Pub. No.: US 2008/0249445 A1
(43) Pub. Date: Oct. 9, 2008

(54) Splint Device

(76) Inventor: Ken Bailey, Victoria (AU)

Correspondence Address: BROOKS KUSHMAN P.C., 1000 TOWN CENTER, TWENTY-SECOND FLOOR, SOUTHFIELD, MI, 48075, US

(21) Appl. No.: 12/065,925

(22) PCT Filed: Sep. 6, 2006
(86) PCT No.: PCT/AU2006/001298

§ 371 Date: Mar. 6, 2008

(30) Foreign Application Priority Data
Sep. 6, 2005 (AU) 2005904859

(51) Int. Cl. A61F 5/00

(52) U.S. Cl. 602/6; 602/5

(57) ABSTRACT

A splint device which provides a compact and versatile flexible device for adequately protecting a person's limb without causing damage by immobilising the injured limb to facilitate healing until further medical attention can be sought. The splint device is inexpensive and can be adjustable in size to fit an individual properly to achieve proper immobilisation of the limb and allow adequate circulation to the damaged area.

### TECHNICAL AREA

The invention relates to the area of medical aids and in particular, to a splint device which provides a compact and versatile flexible device for adequately protecting a person's limb without causing damage by immobilising the injured limb to facilitate healing until further medical attention can be sought.

### BACKGROUND TO THE INVENTION

When a person incurs a serious injury to a limb such as a fractured or broken bone, it is often necessary to immobilise that particular limb until further medical attention can be sought. Over the years there have been a number of attempts at improving splints that are used to immobilise human and animal limbs.

At present the main types of splints available include plastic inflatable sleeves, polyurethane press-type splints and stick-type splints. However, each of these prior art splints have problems and disadvantages associated with them. Whilst the plastic, inflatable sleeve splints are inexpensive, they are fairly bulky and tend to apply pressure to broken areas which can cause additional pain to the casualty and possibly worsen the condition of the injury. In addition, since the splint is made of an inflatable plastic material, it is often prone to leaks or not having sufficient air pressure to function properly.

The polyurethane press-type splints and stick-type splints are disadvantageous in that they are unable to provide adequate support to the entire limb. The press-type splints are difficult to wrap around a limb. The stick-type splints, which are usually made of a visyboard material, only provide one flat contact point for supporting the limb.

Accordingly, it is an object of the present invention to overcome or substantially ameliorate the disadvantages of the prior art by providing a compact and versatile flexible splint device which is adapted to adequately protect a person's limb without causing further damage by immobilising the injured limb to facilitate healing until further medical attention can be sought.

It is a further object of the invention to provide a splint device which is inexpensive and can be adjustable in size to fit an individual properly to achieve proper immobilisation of the limb and allow adequate circulation to the damaged area.

### SUMMARY OF THE INVENTION

The present invention provides a splint device including a body portion, said body portion having a plurality of corrugations, and a securing means for extending around a limb to secure the splint device to the limb whereby, the splint device provides a compact and versatile flexible device for adequately protecting a person's limb without causing further damage by immobilising the injured limb to facilitate healing until further medical attention can be sought.

It is preferred that the splint device is preferably made of a flexible sheet material.

It is preferred that the corrugations form a plurality of parallel ridges and troughs created by the folding of the body portion of the splint device.

It is further preferred that the parallel ridges extend upwardly relative to the horizontal axis of the body portion of the splint device, whilst the parallel troughs preferably extend downwardly relative to the horizontal axis of the body portion of the splint device.

It is further preferred that the edge of each parallel ridge and/or trough is perforated.

It is preferred that the corrugations have provided at least one aperture.

It is further preferred that the aperture(s) is evenly spaced apart from each neighbouring aperture on the same corrugation to provide an even distribution of force or pressure when the splint device is secured around a person's limb.

It is preferred that the securing means is a strip member made of a semi-rigid elastic material.

In order that the invention may be more readily understood we will describe by way of non-limiting example of a specific embodiment thereof.

### BRIEF DESCRIPTION OF THE DRAWING FIGURES

FIG. 1 shows a top perspective view of the splint device in a fully extended flat configuration according to a preferred embodiment of the invention.

FIG. 2 shows a top perspective view of the splint device in a compacted configuration according to a preferred embodiment of the invention.

FIG. 3 shows a side perspective view of the splint device according to a preferred embodiment of the invention in a rolled configuration as it would appear when in use.

FIG. 4 shows a top perspective view of the splint device according to a preferred embodiment of the invention when wrapped around a person's arm.

### DESCRIPTION OF A PREFERRED EMBODIMENT OF THE INVENTION

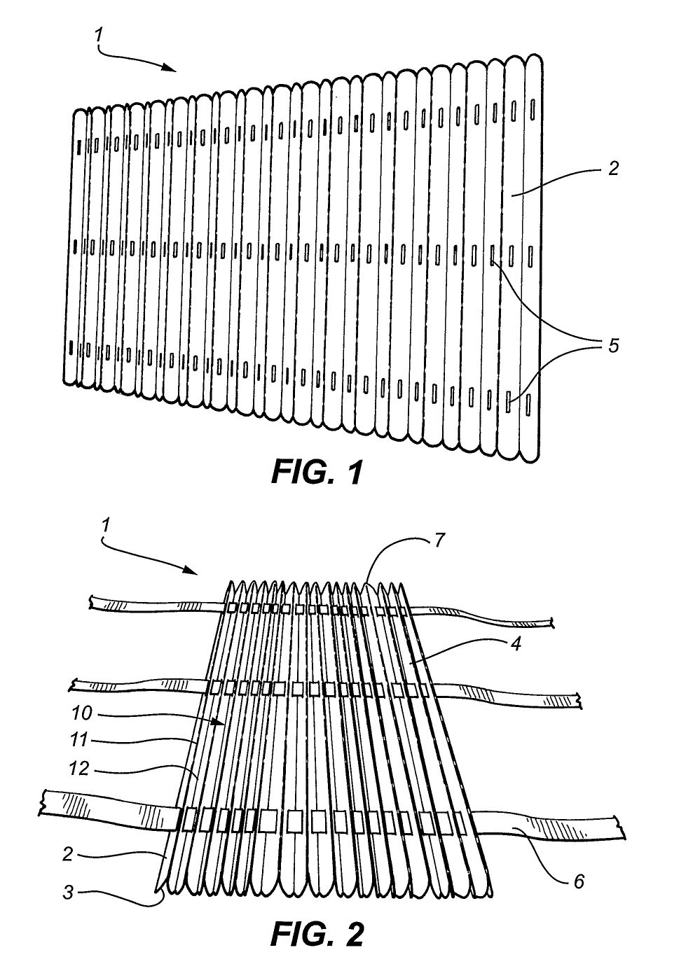

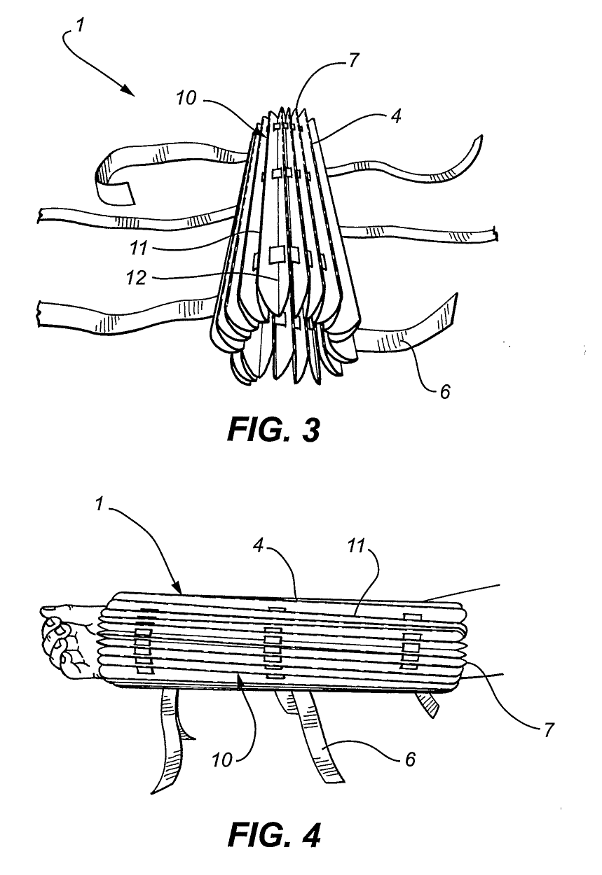

FIGS. 1 to 4 show the splint device according to a preferred embodiment of the invention.

In this preferred embodiment, the invention provides a splint device 1 in the form of a compact and versatile flexible device for adequately protecting a person's limb without causing further damage by immobilising the injured limb to facilitate healing until further medical attention can be sought. The splint device 1 is designed to be inexpensive and adjustable in size to fit an individual properly in order to achieve proper immobilisation of the limb and allow adequate circulation to the damaged area. The splint device 1 is preferably made of a plastics material, board material or any other suitable material which meets the requirements of the invention to provide the device with rigidity, flexibility and cost-effectiveness. It is envisaged that the dimensions, style and colour of the splint device 1 may vary as desired. The splint device 1 is preferably designed to be disposable, particularly in hospital use, although it could also be re-useable upon appropriate cleaning and/or re-sterilisation.

The splint device 1 preferably has provided a body portion 4 which is substantially rectangular in shape when laid in an open, flat configuration. However, it is envisaged that any other suitable shape may also be adopted to enable the device 1 to be better suited for application to particular limbs. The body portion is preferably formed from a single piece of material having a top surface 2 and a bottom surface 3.

When assembled, the body portion 4 of the splint device 1 has provided a plurality of corrugations 10 which is adapted to provide the splint device with a “concertina” shape when it is in an outstretched position. The corrugations 10 are adapted to be evenly positioned along the body portion 4 of the splint device 1 in order to provide even stability and support to the injured body part. However, it is envisaged that the corrugations 10 could also be irregularly spaced and/or have a different thickness of material as desired by a user. The corrugations 10 form a plurality of parallel ridges 11 and a plurality of parallel troughs 12 created by the folding of the body portion 4. The parallel ridges 11 preferably extend upwardly relative to the horizontal axis of the body portion 4 of the splint device 1, whilst the parallel troughs preferably extend downwardly relative to the horizontal axis of the body portion 4 of the splint device 1.

The edge of each ridge 11 and trough 12 of the body portion may preferably be perforated in order to provide additional flexibility to each corrugation 10 and assist in being able to achieve a tighter fold in order to compress the splint device 1 from an expanded configuration when in normal use to a compressed, compact configuration for storage or carrying.

The corrugations 10 of the body portion 4 of the splint device 1 preferably have provided with at least one aperture 5, which is adapted to receive a securing means 6 therethrough. The aperture(s) 5 is adapted to be centrally located along the width of each corrugation 10 and evenly spaced apart from each neighbouring aperture 5 on the same corrugation 10 to provide an even distribution of force or pressure when the splint device 1 is secured around a person's limb. In a preferred embodiment, the aperture(s) 5 is rectangular in shape. However, it is envisaged that any other suitable shape may be employed so long as it is capable of suitably receiving the securing means 6 therethrough and will not hinder performance of the securing means 6. In addition, the size of the aperture(s) may also be variable to suit an alternate securing means 6. The aperture(s) 5 is positioned in an identical position on each corrugation 10 such that when the splint device 1 is in a compressed, compact configuration the opening/passage provided by the aperture(s) 5 extends across the corrugations 10 throughout the entire splint device 1.

The splint device 1 has provided at least one securing means 6 which is adapted to be received through at least one aperture 5 provided on the body portion 4 of the splint device 1. In a preferred embodiment, the securing means 6 is a strip member made of a semi-rigid material with elasticity properties, such as rubber, elastic or the like, which can be used to secure the splint device 1 to the injured limb. The securing means 6 is adapted to be passed through the splint device 1 through each corresponding aperture 5 provided on adjacent corrugations 10 of the body portion 4 of the splint device 1 until each end of the securing means 6 is extending outwardly from either side of the body portion 4 of the splint device 1. The strip member 6 can then be tightened by the desired amount around a person's limb and the ends tied together to secure the splint device 1 around a person's limb. Alternatively, the ends of the strip could be held within a fastener or retention device (not shown) or secured using any other suitable mechanism. The strip member(s) can be easily removed and replaced, allowing the splint device to be adjusted to compensate for swelling of a portion of the injured extremity without the need to remove the entire splint device 1. It is envisaged that the securing means could also be made of elastic, plastic, Velcro (Registered Trade Mark), or any other suitable material. In addition, any other suitable fastening means may also be adopted.

In practice, the splint device 1 is pulled into an extended configuration such that the corrugations 10 of the body portion 4 are spread out like a fan. The “concertina” shape allows the splint device 1 to extend to fit any sized limb. The body portion 4 of the splint device 1 is then wrapped around a person's injured limb and each opposite end of the securing means 6 is pulled and fastened by tying the opposite ends into a knot to secure the splint device 1 around the injured limb. Due to the plurality of corrugations 10 provided along the body portion 4 of the splint device 1, the splint device 1 has provided multiple contact points with the limb to provide better support and restriction of movement of that limb. The long “concertina” shape also provides the splint device 1 with greater longitudinal rigidity than the splints of the prior art. Once medical attention has been sought, the splint device 1 can either be re-used or thrown away. As these splints are inexpensive, they would provide an excellent disposable aid for hospitals which could be packaged, sterilized and thrown away once used.

It is envisaged that one size 7 end of the body portion 4 of the splint device 1 may be tapered such that, the more than one splint device can be connected together in the instance where a longer splint is needed. In this case, the corrugations 10 of the tapered end (not shown) of the splint device 1 can be fitted under and interlocked and/or joined with the corrugations 10 of a complimentary wider end (not shown) of another adjacent splint device 1 to provide a longer splint. The two splint devices 1 will remain in connection with each other upon tightening of the securing means 6 around an injured limb. In this embodiment, the aperture 5 provided at the tapered end of the splint device 1 may be narrow to better accommodate the taper in the body portion 4 of the splint device 1.

While we have described herein a particular embodiment of the splint device, it is further envisaged that other embodiments of the invention could exhibit any number and combination of any one of the features previously described. However, it is to be understood that any variations and modifications can be made without departing from the spirit and scope thereof.

1. A splint device including a body portion, said body portion having a plurality of corrugations, and a securing means to secure the splint device to a limb whereby, the splint device provides a compact and versatile flexible device for adequately protecting a person's limb without causing further damage by immobilising the injured limb until further medical attention can be sought.

2. A splint device as claimed in claim 1 wherein the splint device is preferably made of a flexible sheet material.

3. A splint device as claimed in claim 1 wherein the corrugations form a plurality of parallel ridges and troughs created by the folding of the body portion of the splint device.

4. A splint device as claimed in claim 3 wherein the parallel ridges extend upwardly relative to the horizontal axis of the body portion of the splint device, whilst the parallel troughs preferably extend downwardly relative to the horizontal axis of the body portion of the splint device.

5. A splint device as claimed in claim 3 wherein the edge of each parallel ridge and/or trough is perforated.

6. A splint device as claimed in claim wherein the corrugations have provided at least one aperture.

7. A splint device as claimed in claim 6 wherein the aperture(s) is evenly spaced apart from each neighbouring aperture on the same corrugation to provide an even distribution of force or pressure when the splint device is secured around a persons limb.

8. A splint device as claimed in any claim 1 wherein the securing means is a strip member.

9. A splint device as claimed in claim 8 wherein the strip member is made of a semi-rigid elastic material.

10. A splint device as claimed in claim 8 is fastened by securing the two opposing ends together.

11. (canceled)
